(19) United States
(12) Patent Application Publication
(10) Pub. No.: US 2006/0293668 A1
(43) Pub. Date: Dec. 28, 2006

(54) Bone screw locking mechanism and method of use

(75) Inventors: Jason M. May, Memphis, TN (US); Robert A. Farris, Cordova, TN (US)

Correspondence Address: COATS & BENNETT, PLLC, P O BOX 5, RALEIGH, NC, 27602, US

(73) Assignee: SDGI Holdings, Inc.

(21) Appl. No.: 11/150,506

(22) Filed: Jun. 10, 2005

(51) Int. Cl. A61F 2/30

(52) U.S. Cl. 606/69

(57) ABSTRACT

Devices and methods to prevent a screw from backing out of a plate. The plate includes an aperture for receiving a screw that is attached to the bone. A mount is positioned adjacent to the aperture and extends out from a face of the plate. A locking mechanism is attached to the mount and sized to extend over at least a portion of the inserted screw to prevent the screw from backing out of the aperture. One method comprises inserting a screw into the aperture to attach the plate to the bone. A locking mechanism is then attached to the mount. The locking mechanism is sized to extend over bone screw and prevent the screw from backing out.

### BACKGROUND

Screws are often used for securing a device within a patient, such as screws for connecting a plate to vertebral members. The device includes apertures for receiving the bone screws. The device is positioned within the body and the bone screws are inserted through the apertures and into the bone to fixedly connect the device. One potential issue with attaching a device with screws is the tendency for the screws to back out of the bone. The backing out occurs at some point in the future after the screws are initially inserted into the bone. Correction usually requires another surgical procedure to either re-tighten the screws to the bone, or removal and replacement of the screws and/or device.

An anti-back out device should be straight-forward to use by a physician. This may include making the device in a manner to facilitate attachment to the plate. Further, the device should be constructed such that the physician can determine that it is properly installed and will effectively prevent the back out of the screw.

### SUMMARY

The present application includes devices and methods to prevent a screw from backing out of a plate. The plate includes an aperture for receiving a screw to attach the plate to the bone. A mount is positioned adjacent to the aperture and extends out from a face of the plate. A locking mechanism is attached to the mount and sized to extend over at least a portion of the inserted screw to prevent the screw from backing out of the aperture.

One method comprises inserting a screw into the aperture and attaching the plate to the bone. A locking mechanism is then attached to the mount. The locking mechanism is sized to extend over bone screw and prevent the screw from backing out. The locking mechanism may extend over a single screw, or may extend over a number of screws.

### BRIEF DESCRIPTION OF THE DRAWINGS

FIG. 1 is a perspective view of a plate having a plurality of apertures and mounts according to one embodiment of the present invention;

FIG. 2 is an exploded view illustrating a locking mechanism according to one embodiment of the present invention;

FIG. 3 is a cross-section view of the locking mechanism illustrated in FIG. 2;

FIG. 4 is an exploded view illustrating a locking mechanism according to one embodiment of the present invention;

FIG. 5 is a cross-section view of the locking mechanism illustrated in FIG. 4;

FIG. 6 is an exploded view illustrating a locking mechanism according to one embodiment of the present invention;

FIG. 7 is a cross-section view of the locking mechanism illustrated in FIG. 6;

FIG. 8 is an exploded view illustrating a locking mechanism according to one embodiment of the present invention;

FIG. 9 is a cross-section view of the locking mechanism illustrated in FIG. 8;

FIG. 10 is an exploded view illustrating a locking mechanism according to one embodiment of the present invention;

FIG. 11 is a cross-section view of the locking mechanism illustrated in FIG. 10; and

FIG. 12 is a side view of a mount according to one embodiment of the present invention.

### DETAILED DESCRIPTION

The present application is directed to embodiments of a device and method of use to prevent a screw from backing out of a plate after installation within the body. The plate 20 includes an aperture 21 for receiving a screw 50 that attaches to the bone. A mount 30 is positioned adjacent to the aperture 21 and extends out from a top face of the plate 20. A locking mechanism 40 is attached to the mount 30 and sized to extend over at least a portion of the screw 50 to prevent the screw 50 from backing out of the aperture 21.

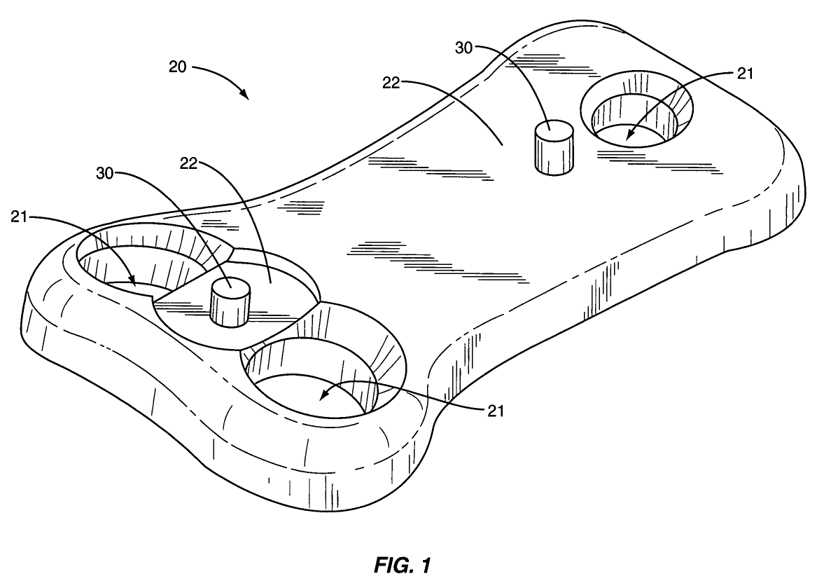

FIG. 1 illustrates one embodiment of the plate 20 having a plurality of apertures 21. A bottom surface of the plate 20 is positioned facing towards the bone, and a second side faces outward from the bone. The plate 20 has an elongated length to span across two or more bones. Apertures 21 may be grouped in sets of two or more as illustrated on a first end of the plate 20 of FIG. 1 (i.e., the left end as illustrated in FIG. 1), or may be isolated as illustrated in the second end of the plate 20 of FIG. 1. A recess 22 may be located adjacent to one or more apertures 21 and extend into the aperture 21. The recess 22 has a depth to contain all or a portion of the locking mechanism 40 as will be explained in detail below. Mounts 30 may be positioned adjacent to the aperture 21 for mounting the locking mechanism 40. Mounts 30 may be positioned within a recess 22, or may extend from a non-recessed surface.

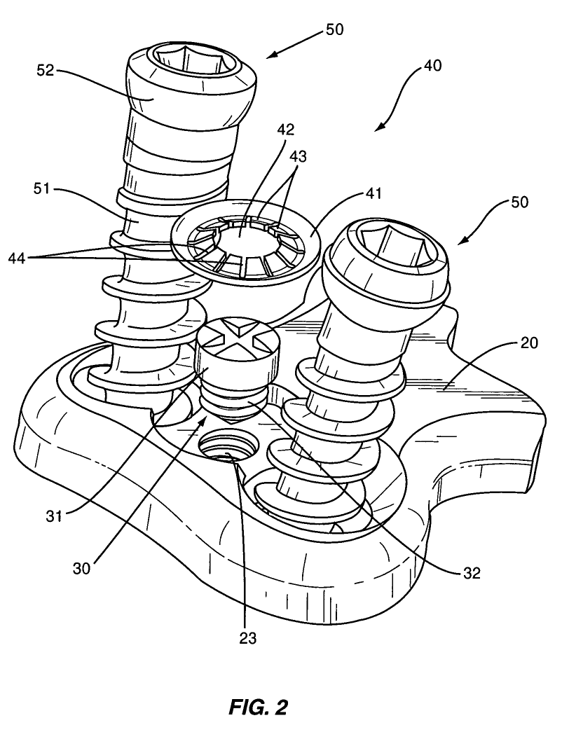

Mounts 30 provide an attachment mechanism for connecting the locking mechanism 40 to the plate 20. FIG. 2 illustrates one embodiment of the mount 30 having a head 31 and a shaft 32. The shaft 32 is threaded and mates within a threaded opening 23 in the plate 20. Opening 23 may extend through the entire thickness of the plate 20 (i.e., extending through the top and bottom surfaces), or may have a depth less than the plate thickness. Once mounted, the head 31 extends above the surface of the recess 22 for receiving the locking mechanism 40. In one embodiment, the head 31 extends above the surface of the plate 20.

One or more screws 50 attach the plate 20 to bone. Screws 50 include a threaded shaft 51 and a head 52. The shaft 51 is sized to fit within the aperture 21. Head 52 is sized to contact the edges of the aperture 21. In one embodiment, aperture 21 has a larger diameter at the top surface of the plate 20 and a smaller diameter at the bottom surface. The smaller diameter is large enough to allow passage of the shaft 51, and restricts passage of the head 52. In one embodiment, head 52 is shaped to conform to the shape of the tapered section. The same type of screws 50 may be inserted in each of the apertures 21 to attach the plate 20 to the bone, or a combination of different types of screws 50 may be used for attachment as is explained in more detail below.

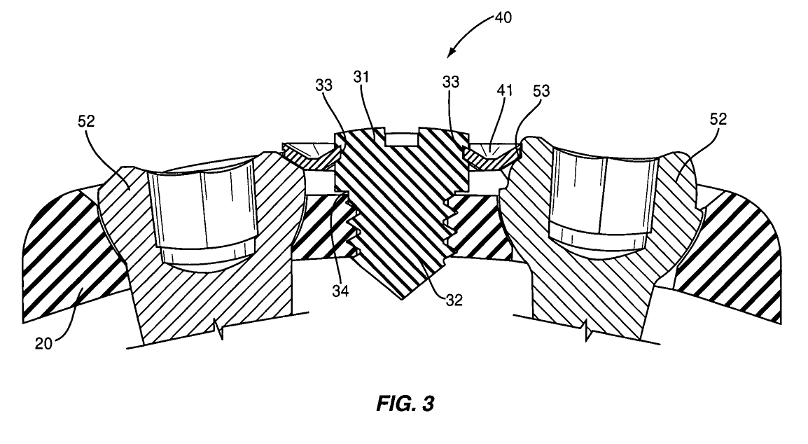

One embodiment of a locking mechanism is illustrated in FIGS. 2 and 3. Locking mechanism 40 comprises a retainer 41 having an outer edge sized to extend over a portion of the screws 50. The retainer 41 may be constructed of a resilient material that deflects when attached to the mount 30. A central opening 42 of the retainer 41 is sized to extend over the mount head 31. Opening 42 is slightly smaller than the head 31 causing the retainer 41 to deflect as it is advanced onto the head 31. The deflection causes the retainer 41 to apply a holding force that is adequate to maintain the retainer 41 attached to the head 31.

Head 31 may further include a notch 33 that extends around a section or entirety of the periphery and spaced a distance from the top of the head 31. The notch 33 is sized to receive the inner edge of the retainer 41. The compressive force applied by the retainer 41 causes the retainer inner edge to seat within the notch 33. In another embodiment, a junction 34 is formed at the intersection of the head 31 and shaft 32. The junction 34 may also be used for receiving the inner edge of the retainer 41 in the same manner as the notch 33.

A series of tabs 43 formed by slots 44 may extend around a portion of the entirety of the inner edge of the opening 42. As illustrated in FIG. 3, retainer 41 may have an angled cross-section shape. Both the tabs 43 and the angular cross-sectional shape facilitate the retainer deflection during insertion onto the head 31.

The method of using this embodiment may vary depending upon the specific application. The plate 20 with mount 30 is initially positioned within the patient and screws 50 are inserted through the apertures 21 to attach to the plate 20 to the bone. The retainer opening 42 is aligned with the head 31 and advanced downward onto the head 31 causing the size of the opening 42 to increase and apply a compressive force to the head 31. In embodiments with a notch 33, or junction 34, the retainer 41 may be advanced until the inner edge of the retainer seats with the notch 33 or junction 34.

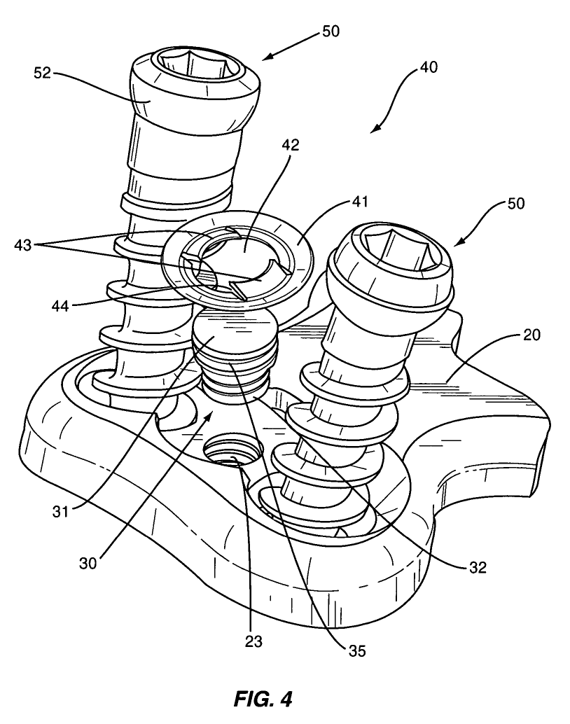

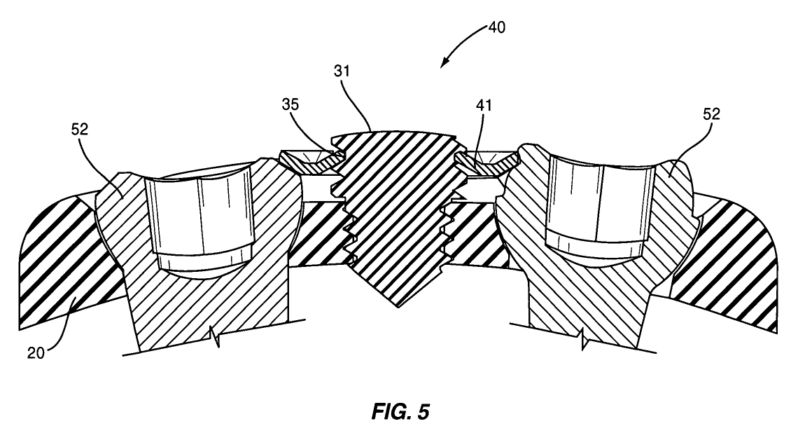

Another embodiment of a locking mechanism 40 is illustrated in FIGS. 4 and 5. Locking mechanism 40 comprises a retainer 41 having a central opening 42. One or more tabs 43 and slots 44 are positioned along the edge of the opening 42. Mount head 31 includes threads 35 to receive the tabs 43. Opening 42 is slightly smaller than the head 31 causing the retainer 41 to deflect as it is advanced onto the head 31. The deflection causes the retainer 41 to apply a compressive force that is adequate to maintain the retainer 41 attached to the head 31. In addition, the inner edges of tabs 43 seat within the threads 35. Retainer 41 may have an angular cross-sectional configuration as illustrated in FIG. 5 to further aid in the deflection.

The tabs 43 further form receiving indents for attachment with a tool to further rotate the retainer 41 further onto the threads 35. Therefore, the threads 35 capture the edges of the opening 42 to prevent the retainer 41 from being removed from the mount 30, and also allow for rotation of the retainer 41 to further attach the retainer 41 to the mount 30.

In use, the plate 20 is positioned within the patient and screws 50 are inserted for attachment to the bone. The retainer 41 is then advanced onto the mount 30 with the opening 42 being deflected to fit onto the mount head 31. At this point, the retainer 41 may or may not be in contact with the screws 50. The retainer 41 may be further rotated onto threads 35 on the head 31 to further move the retainer 41 onto the head 31. The retainer 41 may be rotated with a tool that is attached to the retainer, or by hand by the physician. In one embodiment, retainer 41 is rotated about the mount 30 until it contacts and applies a downward holding force to the screws 50.

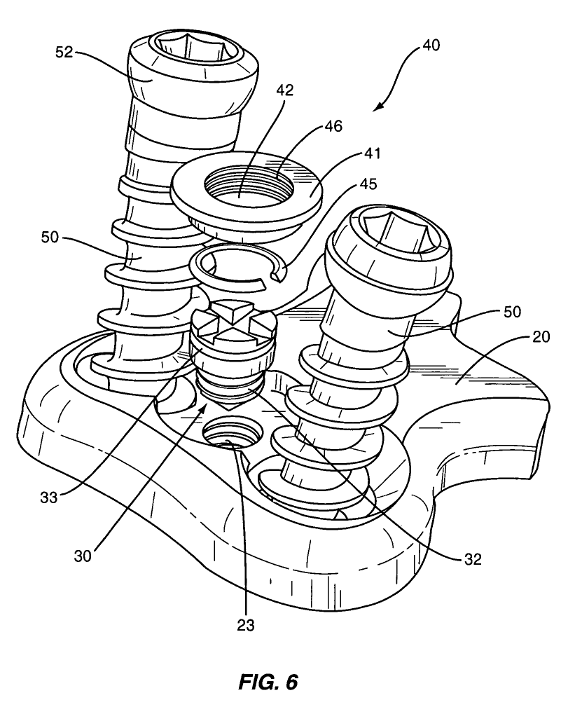

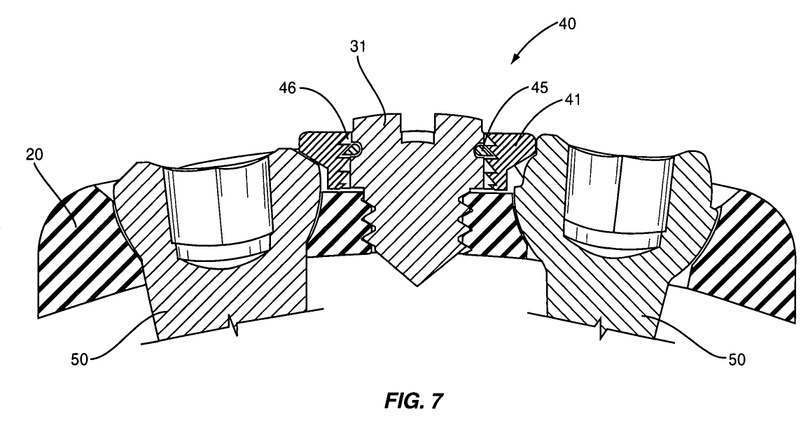

Another embodiment of a locking mechanism 40 is illustrated in FIGS. 6 and 7. Retaining mechanism 40 comprises a ring 45 and retainer 41. Ring 45 forms a connection point between the retainer 41 and the mount 30. In one embodiment, ring 45 is a snap-ring having a C-shaped body that is deflected to fit around and apply a compressive force to the head 31. To further maintain the ring 45 positioned on the head 31, head 31 may include a notch 33 sized to receive an inner edge of the ring 45. Notch 33 further prevents the ring 45 from sliding along the head 31 when the retainer 41 is attached.

Retainer 41 includes a central opening 42 sized to fit around the head 31. Threads 46 extend around the inner edge of the opening 42 and interact with the outer edge of the ring 45 when the retainer 41 is attached to the mount 30. Retainer 41 may be constructed of a deflectable material, or a rigid material depending upon the application.

In use, the ring 45 may be attached to the mount 30 prior to the plate 20 being inserted into the patient. The plate 20 is placed in the patient and screws 50 are inserted to attach the plate 20 to the bone. The retainer 41 is then advanced onto the mount 30 and connected by the ring 45. In one embodiment, retainer 41 is a rigid material and the ring 45 is deflected inward into the notch 33 as the retainer 41 is advanced onto the mount 30. When a thread 46 aligns with the ring 45, the ring 45 rebounds outward towards its original shape and seats within the thread 46. As best illustrated in FIG. 7, and inner edge of the ring 45 seats within the notch 33 and an outer edge within a thread 46. In another embodiment, both the retainer 41 and the ring 45 deflect as the retainer 41 is advanced onto the mount 30. The ring 45 may also be initially attached to the retainer 41 and both are advanced onto the mount 30 in a similar technique as described above.

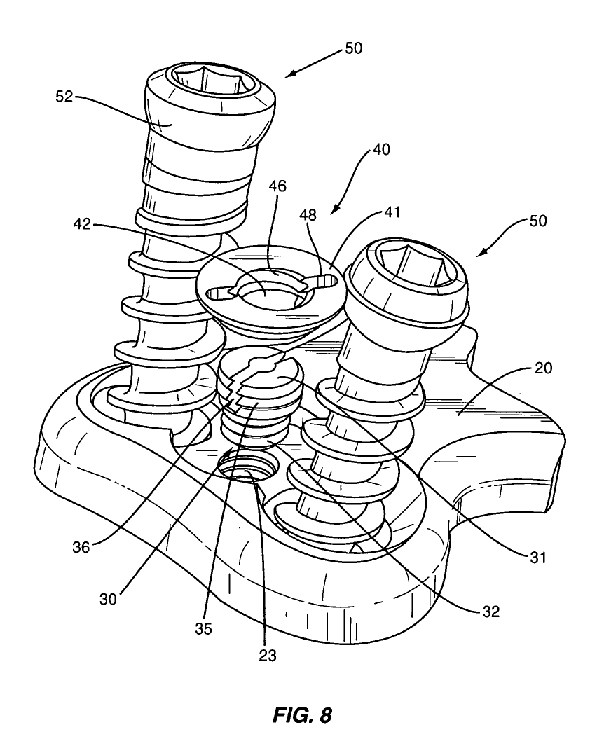

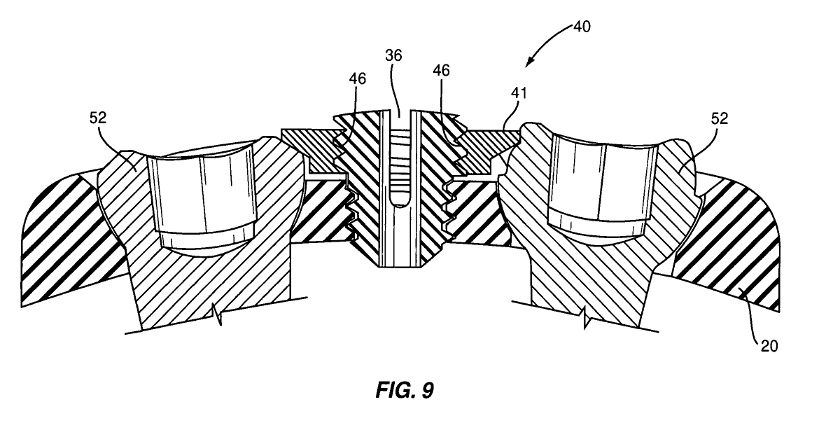

FIGS. 8 and 9 illustrate another embodiment of the mount 30 and locking mechanism 40. Mount 30 includes a split head 31 having first and second sections separated by a gap 36. Gap 36 may have a depth that extends through only a section of the head 31, the entirety of the head 31, or into the shaft 32. Threads 35 are further positioned on the exterior of the head 31. Locking mechanism 40 includes a retainer 41 having a central opening 42 with threads 46. During use, retainer 41 is advanced onto the head 31 causing the head 31 to compress inward thereby reducing the width of the gap 36. The force applied by the retainer 41 is advanced onto the head a distance, and then additionally may be rotated to further move onto the mount and apply a holding force to the screws 50. Rotation of the retainer 41 is caused by a tool that mounts within the receiver 48.

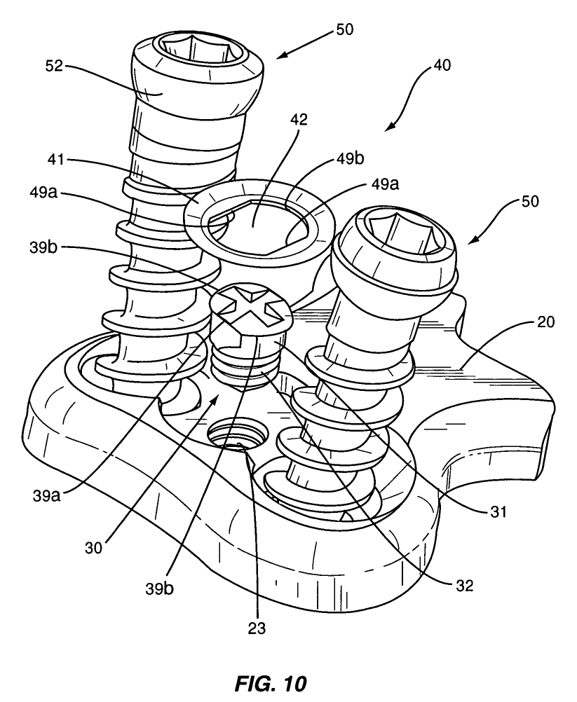

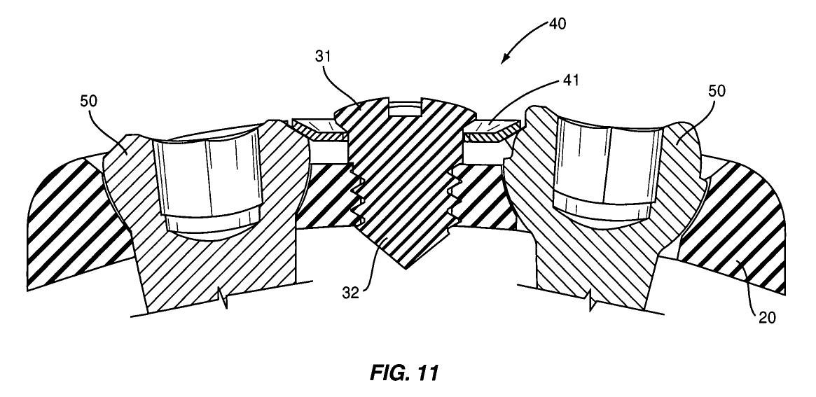

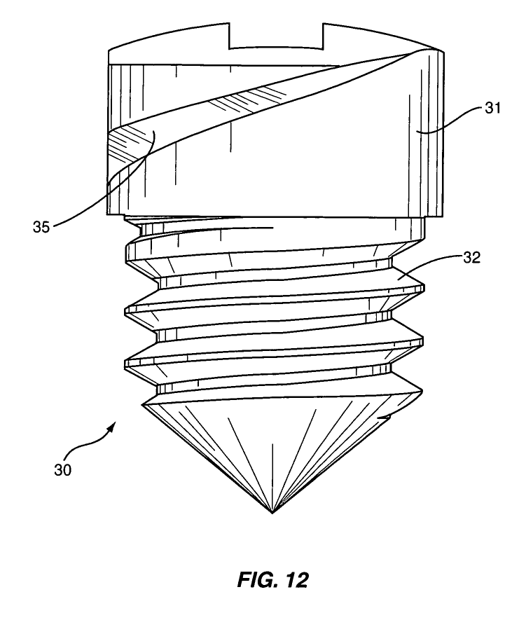

Another mount 30 and locking mechanism 40 embodiment is illustrated in FIGS. 10, 11, and 12. Mount 30 includes a single thread 35 that extends around the head 31. The head 31 has a generally elongated shape with first sides 39a being substantially more linear than second sides 39b. Retainer 41 has a central opening 42 having a generally elongated shape that roughly corresponds to the head 31. Opening 42 includes edges 49a being substantially more linear than second edges 49b. In one embodiment, the opening 42 is smaller than the head 31 causing the retainer 41 to deflect when it is advanced onto the head 31.

In use, the screws 50 are inserted to attach the plate 20 to the bone. The retainer 41 is then advanced onto the head 31. The elongated edges 49a of the retainer 41 are generally aligned with the elongated sides 39a of the head 31 with the second edges 49b generally aligning with the second edges 39b. In one embodiment, the opening 42 is smaller than the head 31 and expands to thereby apply a holding force onto the mount 30. The retainer 41 is further rotated with the edges 49a, 49b riding along the thread 35 of the head 31 to further move the retainer 41 onto the mount 30. In another embodiment, opening 42 is the same size or slightly larger than the head 31. The opening 42 is aligned with the head and then rotated causing edges 49a, 49 to ride along the thread 35 and be further pulled onto the head 31.

In the embodiments described above, the mount 30 may be attached to the plate 20 prior to insertion of the plate 20 into the patient. In one embodiment as illustrated in FIG. 1, the plate 20 and mounts 30 are constructed in a single-piece construction. In a two-piece construction, the mount 30 may be attached to the plate 20 during manufacturing, or at the time of the medical procedure.

The retainer 41 may or may not be in contact with the screws 50 when it is attached to the mount 30. In one embodiment, a space may exist between the retainer 41 and top of the screw head 52. In the event the screw 50 begins to back out of the bone, the screw head 52 makes contact with the retainer 41 which prevents further backing out. In another embodiment, the retainer 41 is in contact with the screw heads 52 when attached to the mount 30. In one mount embodiment, the retainer 41 is advanced onto the mount until the retainer 41 makes contact with the screw head 52. The retainer 41 is then further rotated onto threads on mount head 31 to further move the retainer 41 onto the mount 30 and apply a greater compressive force to the screw head 52. This force holds the screws 50 relative to the plate 20 and prevents back out.

In these embodiments, the physician may be able to visually observe the location of the retainer 41 relative to the head 31. This visual confirmation ensures that the retainer 41 is adequately attached to the mount 30 and extends over the screws 50 to prevent back out. The physician may also have tactile feedback to ensure proper mounting of the retainer 41. The feedback may include feeling the expansion of the opening 42 as it is deflected over the mount head 31 such as the embodiments of FIG. 2 and the deflection of the tabs 43 as in the embodiment of FIG. 4. Tactile feedback may also be felt by the snapping action as the outer edge of the ring 45 seating within the retainer threads 46 of the embodiment of FIG. 6, or the ring 41 seating within the thread 35 of FIG. 10. Tactile feedback may also occur as the retainer 41 is advanced down onto the screw heads 52.

While the retainer 41 is mounted onto the mount 30, the screws 50 may not be able to be removed from the bone. In some embodiments, removal requires the locking mechanism 40 to be removed from the mount 30. This may occur by the retainer 41 being unthreaded from the mount 30, or otherwise removed. Once the retainer 41 is removed, the screws 50 can be removed from the bone. In other embodiments, the mount 30 is unthreaded from the plate 20 causing the retainer 41 to be lifted away from the screws 50.

In some embodiments, a receiver 48 is positioned on the top surface of the retainer 41 for receiving a tool to rotate the retainer 41 as illustrated in FIG. 8. Receiver 48 may include a single tool receiving section, or may include offset tool receiving sections on opposing sides of the opening 42.

In several embodiments described above, the retainer 41 is threadingly engaged with the mount head 31. In another embodiment, one or both the retainer 41 and mount head 31 include a series of parallel indents and teeth instead of threads 46. The retainer 41 is advanced onto the mount head 31 with the indents and teeth causing a ratcheting action as the retainer 41 mates with the head 31. By way of example using the embodiment of FIG. 7, retainer 41 includes a series of teeth and indents that are sized to contain an outer edge of the ring 45. As the retainer 41 is advanced onto the mount head 31, ring 45 is compressed inward as the teeth pass, and then expand outward into the indents.

The mount 30 is positioned for the retainer 41 to extend over a portion of the screws 50. The portion may include the top surface of the screw head 52, or some other section of the screw 50. By way of example, FIG. 3 illustrates the retainer 41 extending over a lip 53 on the edge of the screw head 52. The amount that the retainer 41 extends over the screw 50 is adequate to prevent the screw from backing out of the bone. This may include a limited amount of overlap, or an extensive amount.

Retainer 41 may be positioned to extend over a single screw 50, or a plurality of screws 50. In the embodiment of the right end of FIG. 1, mount 30 is positioned to receive a retainer 41 that extends over the single aperture 21. In other illustrated embodiments, mount 30 is positioned for the retainer 41 to extend over two or more apertures 21.

The mount 30 may be positioned relative to two apertures 21 for the retainer 41 to extend over portions of two or more screws 50. In one embodiment, mount 30 is centered between the plurality of apertures 21 for the retainer 41 to extend an equal amount over each of the screw heads 52. In another embodiment, mount 30 is positioned a different distance away from the apertures 21 and the retainer 41 does not extend an equal amount over each screw head 52.

The mounts 30 may be attached to the plate 20 in a variety of manners. In one embodiment, mount 30 includes a threaded shaft 32 that engages an opening 23 in the plate 20. Mounts 30 may also be attached through adhesives, welding, snap locks, etc. Further, the different mounts 30 attached to a single plate 20 may be attached by different means. By way of example, a first mount may be attached by a threaded shaft 30, and a second mount may have a compressible shaft with feet extending from a distal end that snap against the bottom surface of the plate 20.

Retainer 41 may have an angular cross-sectional shape. This shape may facilitate deflection during insertion, both by allowing expansion of the retainer opening 42, and bending as the retainer 41 is compressed onto the screw head 52. In another embodiment, retainer 41 is substantially flat.

One application for the plate 20 and locking mechanism 40 is for attachment of two or more vertebral members. Other applications are also possible and considered within the scope of the embodiments.

A variety of screws 50 may be used in these embodiments, such as a fixed angle screw and a variable angle screw as illustrated in FIGS. 2 and 3. Fixed angle screw (illustrated on the left side of the plate 20) includes an intermediate section between the head 52 and shaft 51. The head 52 includes a top surface and a spherical surface between the top surface and the intermediate section. The fixed angle screw 50 extends through the aperture 21 with the spherical surface contacting corresponding spherical surfaces of the aperture 21. Once the screw 50 is completely seated, the diameter of the intermediate portion approximates the width of the aperture to provide a snug relationship so that the screw 50 is not able to pivot or translate relative to the plate 20. Variable angle screw (illustrated on the right side of the plate 20) also includes an intermediate section positioned between the head 52 and shaft 51. Head 52 includes a spherical surface disposed between the top surface and the intermediate section. The intermediate section of the variable angle screw is narrower than that of the fixed angle screw. Once seated with the aperture 21, the intermediate section is spaced inward from the edges of the aperture 21. Thus, with the spherical surface of the head seated within the corresponding spherical surface of the aperture 21, the variable angle screw 50 can be angulated relative to the plate 20 and to an axis of the aperture 21. The degree of angulation is dictated by the size of the intermediate section and the size of the aperture 21. Further, the screw 50 can be delivered at a variety of angles relative to the axis of the aperture 21. Embodiments of fixed angle and variable angle screws are disclosed in U.S. Pat. No. 6,669,700, and are herein incorporated by reference in their entirety.

In one embodiment, the retainer 41 is attached to the mount 30. The mount 30 with attached retainer 41 is then further rotated into the plate 20 to further advance the retainer 41 relative to the screw 50. For revision, the retainer 41 may remain attached to the mount 30 and detachment of the mount 30 from the plate thereby also removes the retainer 41.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A plate system adapted to connect to a vertebral member comprising:
a plate having a first surface that faces towards the vertebral member and a second surface that faces away from the vertebral member;
an aperture that extends through the plate from the first surface through the second surface;
a screw having a leading end for insertion into the vertebral member and a head opposite from the leading end, the head having a top surface;
a mount extending from the second surface of the plate and positioned a first distance from the aperture; and
a retainer adapted to prevent the screw from backing out of the vertebral member, the retainer having an opening and an outer edge with the opening sized to fit over the mount with the outer edge being positioned over a portion of the screw, the retainer adapted to be attached to the mount after the bone screw has been inserted into the vertebral member.

2. The system of claim 1, wherein the mount further comprises a threaded shaft that attaches with an opening in the plate.

3. The system of claim 1, wherein the mount includes a notch having a reduced width, the notch sized to receive an edge of the retainer.

4. The system of claim 1, further comprising threads extending along at least a section of the mount.

5. The system of claim 1, wherein an opening width is smaller than a mount width.

6. The system of claim 5, wherein the retainer is constructed of a resilient material that deflects when the retainer is placed onto the mount.

7. The system of claim 5, further comprising a plurality of tabs extending into the retainer from an inner edge of the opening.

8. The system of claim 1, wherein the retainer extends over a top surface of the screw.

9. A plate system adapted to connect to a vertebral member comprising:
a plate having a first surface that faces towards the vertebral member and a second surface that faces away from the vertebral member;
first and second apertures that extend through the plate from the first surface through the second surface;
at least one screw having a leading end for insertion into the vertebral member and a head opposite from the leading end;
a mount extending from the second surface of the plate and positioned a first distance from the first aperture and a second distance from the second aperture; and
a retainer adapted to prevent the at least one screw from backing out of the vertebral member, the retainer having an opening and an outer edge, the retainer constructed to deflect during insertion onto the mount to attach the retainer to the mount with the outer edge being positioned over a portion of each of the at least one screw, the retainer adapted to be attached to the mount after the bone screw has been inserted into the vertebral member.

10. The system of claim 9, wherein the retainer further comprises a plurality of tabs and slots aligned on an inner edge of the opening.

11. The system of claim 9, wherein the mount further comprises a notch that receives an inner edge of the opening to attach the retainer to the mount.

12. The system of claim 9, further comprising a ring positioned between the mount and the retainer, the ring functioning to attach the retainer to the mount.

13. The system of claim 12, wherein the ring is positioned within a notch in the mount.

14. The system of claim 9, further comprising a cam mechanism on the outer edge of the mount that receives an inner edge of the opening to attach the retainer to the mount and compress the screws.

15. The system of claim 9, wherein the mount comprises first and second sections separated by a gap.

16. The system of claim 9, wherein the mount is removably attached to the plate, and the mount and the retainer are removable together as a unit from the plate.

17. A method of using a plate system for a vertebral member, the method comprising the steps of:
positioning a mount to extend upward from a first surface of the plate prior to inserting the plate against the vertebral member;
attaching the plate to the vertebral member by inserting a screw through an aperture in the plate and into the vertebral member;
attaching a retainer to the mount; and
positioning a section of the retainer to extend over a portion of the screw to prevent the screw from backing out of the vertebral member.

18. The method of claim 17, further comprising rotating the retainer relative to the mount and applying an additional compressive force to the portion of the screw.

19. The method of claim 17, further comprising deflecting the mount while attaching the retainer.

20. The method of claim 17, wherein the step of positioning the section of the retainer to extend over the portion of the screw to prevent the screw from backing out of the vertebral member further comprises positioning the retainer to extend over a second screw.

21. The method of claim 17, wherein the step of attaching the retainer to the mount comprises positioning a ring between the retainer and the mount.

22. The method of claim 17, wherein the step of attaching the retainer to the mount comprises causing a ratcheting action between the retainer and the mount by advancing the retainer onto the mount.

23. The method of claim 17, further comprising removing the mount and the attached retainer together from the plate and then removing the screw from the vertebral member.
